(19) United States
(12) Patent Application Publication
(10) Pub. No.: US 2014/0088597 A1
(43) Pub. Date: Mar. 27, 2014

(54) METHOD OF INCREASING BONE DENSITY DURING SURGERY

(71) Applicants: TRUSTEES OF BOSTON UNIVERSITY, Boston, MA (US); SOCIETE POUR LA CONCEPTION DES APPLICATIONS DES TECHNIQUES ELECTRONIQUES, Merignac (FR)

(72) Inventors: Serge Dibart, Waltham, MA (US); Robert GYURKO, Reading, MA (US); Robert GREGOIRE, Ludon-Medoc (FR)

(73) Assignees: TRUSTEES OF BOSTON UNIVERSITY, Boston, MA (US); SOCIETE POUR LA CONCEPTION DES APPLICATIONS DES TECHNIQUES ELECTRONIQUES, Merignac (FR)

(21) Appl. No.: 14/037,527

(22) Filed: Sep. 26, 2013

Related U.S. Application Data
(60) Provisional application No. 61/705,746, filed on Sep. 26, 2012.

(51) Int. Cl. A61B 17/16

(52) U.S. Cl. CPC A61B 17/1673
USPC 606/79

(57) ABSTRACT

The present invention concern a method for increasing bone density in a patient in need thereof which comprises the step of cutting the bones of said patient with a piezo ultrasonic surgical knife using a ultrasonic frequency of between 28 000 and 36 000 Hz.

### FIELD OF THE INVENTION

This invention relates to a method of increasing bone density using a piezo ultrasonic surgical knife, in particular during oral surgery, maxillo facial surgery, implantology or orthopedic surgery.

### BACKGROUND OF THE INVENTION

Bone surgery using ultrasonic surgical knife is gaining popularity, in particular in oral and maxillo facial surgery, for their precision and ability to spare soft tissue. In particular piezo driven osteotomes such as Piezotome® are now being used for corticotomy, sinus lift, ridge splitting and implant osteotomy.

In 2009, Dr Dibart described a new minimally invasive procedure that he called Piezocision® (Dibart S, Sebaoun J D, Surmenian J. Piezocision: A minimally invasive, periodontally accelerated orthodontic tooth movement procedure. Compend Contin Educ Dent. 2009;30(6):342-350. 24; Dibart S, Surmenian J, Sebaoun J D, Montesani L. Rapid treatment of Class II malocclusion with piezocision: two case reports. Int J Periodontics Restorative Dent. 2010;30(5):487-493). This technique combines micro-incisions limited to the buccal gingiva that allow for the use of a piezoelectric knife (eg, Piezotome®) to decorticate the alveolar bone and initiate the regional acceleratory phenomenon (RAP). The procedure allows for rapid tooth movement while correcting hard- and soft-tissue deficiencies when needed. Therefore, piezocision is able to achieve rapid orthodontic tooth movement without the downside of extensive and traumatic conventional surgical approaches due to its temporary bone demineralization which allow fast tooth movement. However, teeth retention means are necessary after orthodontic tooth movement in order to avoid that the teeth move back to their initial position, which increases orthodontic treatment time, discomfort, and expenses. The conventional length of the treatment time also leads to low patient acceptance of the treatment.

Furthermore, some surgery such as implantology could not be carried out on a patient having bones of low quality. Indeed, in particular in dental implant, there is high risk that the implant fell from the jaw before its osteointegration. Therefore there is a need to increase implant primary stability and therefore bone density.

High frequency, low amplitude mechanical stimulation with piezo-ultrasonic osteotomy stimulates osteoblast growth (Frias C, Reis J, Capela e Silva F, Potes J, Simões J, Marques A T: Polymeric piezoelectric actuator substrate for osteoblast mechanical stimulation, J Biomech. 2010 Apr. 19;43(6):1061-6. Epub 2010 Feb. 8). However this stimulation required a long time of action.

It is also known to heal bones using an ultrasonic device by periodic (e.g. daily) application of ultrasound in the oral cavity (U.S. Pat. No. 5,496,256). However, such a healing process is only carried out after surgery and is time consuming for the patient since it must be periodically applied and therefore leads to low patient compliance. Furthermore, it will not be enough to allow implantology on patients of bone of low quality or to suppress teeth retentions means in orthodontic tooth movement.

Therefore there is a need of a new method for increasing bone density which will allow bone surgery to be performed on every type of patients, even on the ones having bones of low quality, and which could be performed at the same time of surgery, avoiding any time consuming post treatment and increasing patient acceptance of the surgery.

It has been randomly discovered by the inventors that cutting bones with a piezo ultrasonic surgical knife having a specific frequency increases bone density and induces hypermineralization of the bone at and around the site where the bones has been cut by the ultrasonic surgical knife. This is totally surprising since before this increase of bone density, there is first a marked drop in bone density. The increase in bone density does not occur when bur is used instead of the piezo ultrasonic surgical knife. This new discovered effect of the use of a piezo ultrasonic surgical knife will change the way surgery are performed, the patients types on which surgery can be performed and the proposed post-treatment protocols.

### SUMMARY OF THE INVENTION

Briefly, the present invention provides a method for increasing bone density in a patient in need thereof. Said method comprises the step of cutting the bones of said patient with a piezo ultrasonic surgical knife using an ultrasonic frequency of between 28 000 and 36 000 Hz. In an advantageous embodiment the piezo ultrasonic surgical knife is an ultrasonic osteotome such as the Piezotome® from SATELEC. The cutting of bones using Piezotome® is in general called piezo-ultrasonic osteotomy. The increase in bone density will advantageously occur at the site or near the site (in particular immediately around at 2-3 mm of the site) of bone cutting, and will in an advantageous manner take place a few weeks after cutting of the bones, advantageously at least 8 weeks after cutting the bones (in the animal model).

In a particular embodiment, the patient in need thereof has bones of low quality. The bones of low quality could be due to ageing, bad habits (smoking, alcohol drinking), diseases or conditions in which inducible NO synthase is not inactivated, or a long term treatment. Advantageously, the patient in need thereof is chosen between patients suffering from osteoporosis or diabetes, smokers, alcoholic, patients having type 3 or 4 bones, patients who are undergoing or have undergone long-term administration of corticosteroids, patients suffering from Cushing's syndrome, thyroidal or parathyroidal diseases or conditions and patients having gonadal dysgenesis.

In another embodiment the cutting consist in at least one micro incision of between 3 and 15 mm depth and between 1 and 7 mm wide. In the case of orthodontic tooth movement, the micro-incision will have a depth of 3 mm and a width of between 1 and 5 mm. In the case of tooth implant the micro-incision will depend on the size of the implant and in particular will have in general a 5-7 mm width and a 13-15 mm depth. The micro-incision is therefore performed by the piezo ultrasonic surgical knife.

In a particular embodiment, the cutting of the bones occurred during a therapeutic treatment of a patient in need thereof. The patient in need thereof could be a human or an animal. In an advantageous embodiment it is a human. The therapeutic treatment can consist in a dental treatment, oral treatment, medical or veterinary treatment more advantageously dental treatment. In a further embodiment, the cutting of the bones occurred during surgery. Therefore by performing a brief piezo ultrasonic cutting of the bones during surgery with a piezo ultrasonic surgical knife using a ultrasonic frequency of between 28 000 and 36 000 Hz, it is possible at the same time to enable an increase in bone density which in general will occurred after surgery. Advantageously, the surgery consists in surgery in which the bone density is paramount. In particular, the surgery consists in oral surgery, maxillo facial surgery, implantology, veterinary application, fracture healing, fracture consolidation, treatment of non-union, orthopedic surgery or any surgery intended to correct bone deformities such as facial malformations and limb defects. Maxillo facial surgery can consist in sinus lift, ridge splitting, corticotomy or orthodontic tooth movement. Implantology can consist in implant site preparation with or without bone augmentation, in particular dental implant site preparation, implant osteotomies and dental implant placement. More advantageously the surgery will not be followed by a post-treatment requiring ultrasonic healing therapy such as the one described in U.S. Pat. No. 5,496,256.

In an example of the method according to the present invention in which the cutting of the bones occurred during surgery, the surgery consists in implantology and the patient in need thereof has bones of low quality. Usually in this case it is not possible to insert an implant in a patient having bones of low quality, in particular a tooth implant, since the osteointegration will be of bad quality and there is a high risk of implant instability. In case of tooth implant there is a high risk that the implant will not stay in the jaw. Therefore the clinician in general refuse to perform the operation. However, using the method according to the present invention will enable to perform these acts in any type of patients, even in patients having bones of bad quality. Therefore advantageously in the method according to the present invention the surgery consists in implant site preparation for dental implant with or without bone augmentation and the increase in bone density provides better implant primary stability and earlier prosthetic restoration.

In general, the method according to the present invention in which surgery consists in dental implant surgery is performed as follow: After local anesthesia, an incision is done on the gingiva to allow for reflection of a full thickness flap. Once the bone is exposed an osteotomy is done, using the piezo ultrasonic surgical knife at an ultrasonic frequency of between 28 000 and 36 000 Hz according to the method of the present invention, to the depth and width that has been planned and an implant is inserted into the bone. A few sutures are used to suture the flap afterwards.

In another example of the method according to the present invention in which the cutting of the bones occurred during surgery, surgery consists in oral or maxillo facial surgery, in particular in orthodontic tooth movement. Usually, teeth retention means are necessary after surgery in order to avoid that the teeth will move back to their initial position. However, it is not the case if the method according to the present invention is used.

Therefore in an advantageous embodiment of the method according to the present invention the surgery consists in orthodontic tooth movement and the increase in bone density provide the avoidance of the use of any teeth retention means. In general, the method according to the present invention in which surgery consists in orthodontic tooth movement is as follow: One week after the brackets have been placed on the teeth, and under local anesthesia, small interproximal incisions are done buccally. These incisions will then be used to decorticate the alveolar bone using the piezo ultrasonic surgical knife at an ultrasonic frequency of between 28 000 and 36 000 Hz such as the Piezotome® according to the method of the present invention. Bone or soft tissue may or may not be added. 2 weeks later the orthodontic wire are activated.

### BRIEF DESCRIPTION OF THE DRAWINGS

In the accompanying drawings, certain exemplary modes of carrying out the present invention are shown for illustrative purposes:

FIGS. 1, 2 and 11 show a perspective view, a side view and a front view of a first type of a piezo ultrasonic surgical knife tip (tip A) intended to be used in the method according to the present invention.

FIG. 3 shows a 3-D reconstruction of the mouse maxilla 1 week after piezo-ultrasonic osteotomy. Arrow points to the piezo-ultrasonic osteotomy cut.

FIG. 4 shows changes in bone density over time at the piezo-ultrasonic osteotomy cut site in WT and KO mice.

FIG. 5 shows changes in bone density over time behind piezo-ultrasonic osteotomy cut site (2-3 mm) in WT and KO mice.

FIG. 6 shows changes in bone density 8 weeks after bur osteotomy in WT and KO mice.

FIG. 7 shows direct comparison of changes in bone density at the 8 week timepoint in WT and KO mice after piezo-ultrasonic osteotomy (piezo) or bur osteotomy (bur) expressed as percent of untreated control.

FIG. 8 shows changes in bone volume fraction of the total sample volume (BV/TV) over time at the piezo-ultrasonic osteotomy site in WT and KO mice.

FIG. 9 shows changes in bone volume fraction of the total sample volume (BV/TV) 8 weeks after bur osteotomy in WT and KO mice.

FIG. 10 shows direct comparison of changes in bone volume fraction of the total sample volume at the 8 week timepoint in WT and KO mice after piezo-ultrasonic osteotomy (piezo) or bur osteotomy (bur) expressed as percent of untreated control.

FIG. 12 shows a perspective view of a second type of a piezo ultrasonic surgical knife tip (tip B) intended to be used in the method according to the present invention. This tip is used to do lateral maxillary or mandibular piezocision cuts. Here for the left side of the patient.

FIG. 13 shows a perspective view of a third type of a piezo ultrasonic surgical knife tip (tip C) intended to be used in the method according to the present invention. This tip is used to do lateral maxillary or mandibular piezocision cuts. Here for the right side of the patient.

FIG. 14 shows a perspective view of a fourth type of a piezo ultrasonic surgical knife tip (tip D) intended to be used in the method according to the present invention. This tip is used for upper or lower molar distalization during piezocision as well as computer guided piezocision surgery.

### DETAILED DESCRIPTION OF THE PREFERRED EMBODIMENTS

Other characteristics and advantages of the invention are demonstrated by the following description of particular ways of devising the invention which are given as non-restrictive examples, in reference to the appended drawings.

Referring in details to the drawings, the references numerals herein refer to the like numbered elements in the drawings.

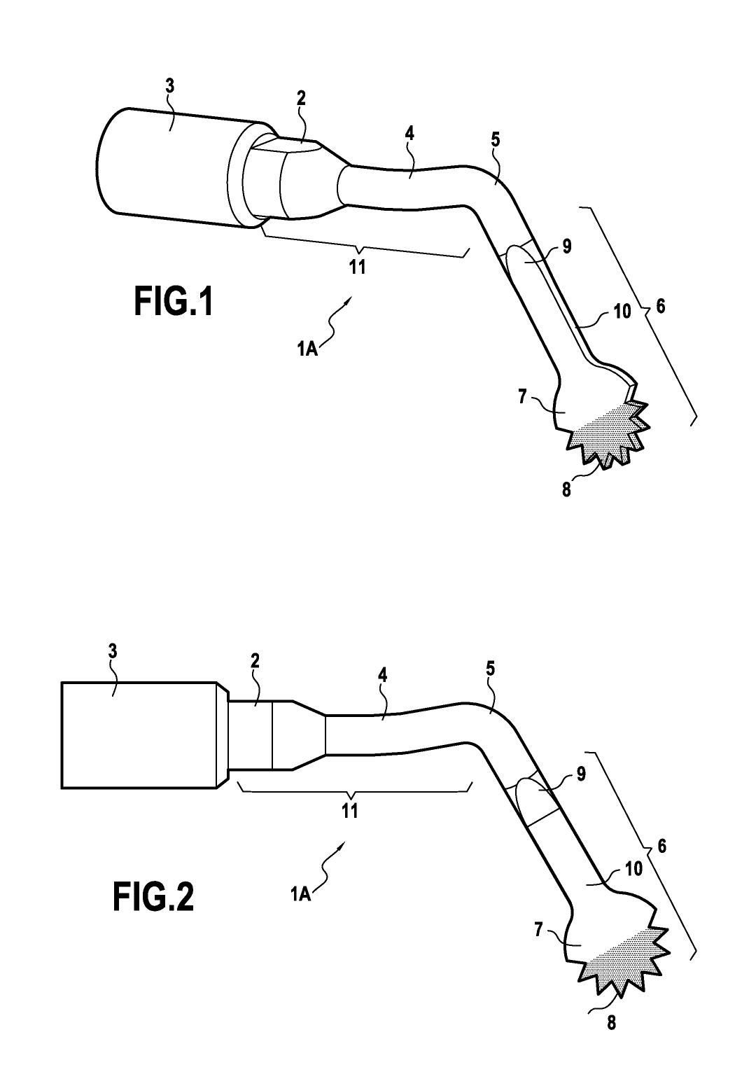

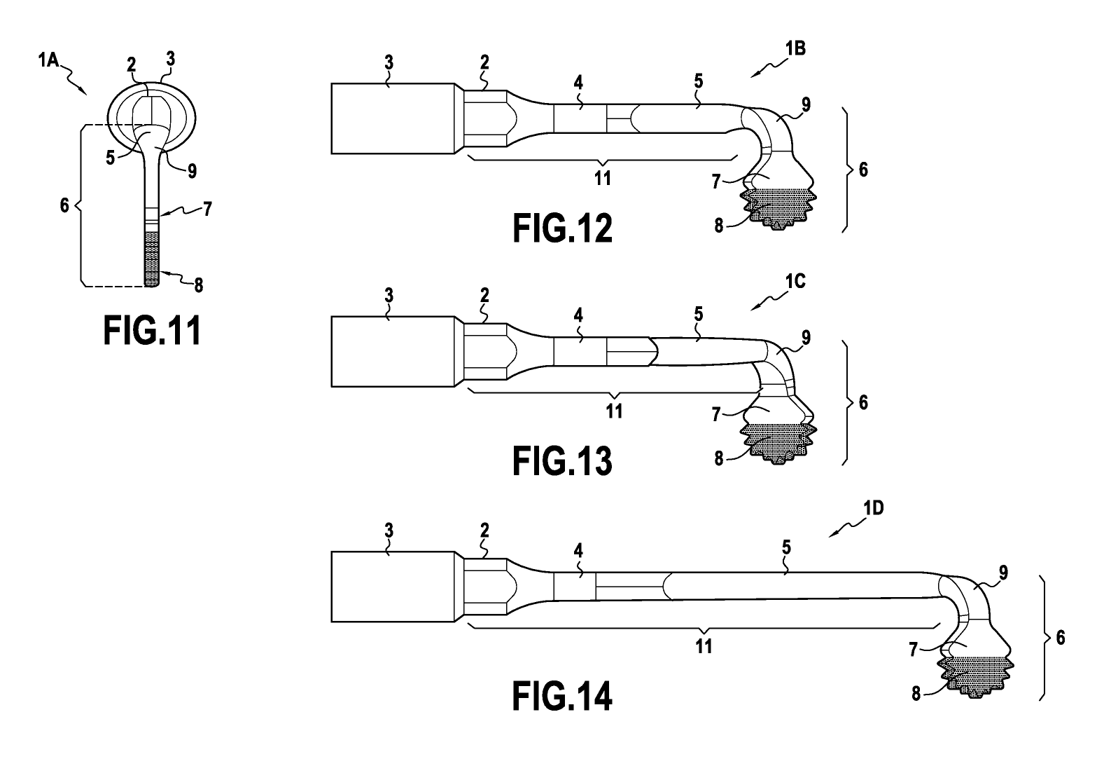

Referring now to FIGS. 1, 2 and 11 there is illustrated a first type of piezo ultrasonic surgical knife tip (tip A) 1_{A} which could be manufactured in stainless steel and/or titanium alloys intended to be used in the method according to the present invention, in particular during maxillo facial or oral surgery, such as orthodontic tooth movement.

The tip 1_{A} has a rectilinear proximal section 3, an operating head 6 located on the free end side of the piezo surgical knife tip and an intermediate portion 11 connecting the proximal section 3 to the operating head 6.

The proximal section 3 has a cylindrical shape being of greater thickness than the other parts of the tip. It is intended to be fitted or screwed or otherwise securely mounted to a cylindrical handpiece (not shown) of the piezo ultrasonic surgical knife acting as a handle for the surgeon. The handpiece is part of an ultrasonic equipment which will vibrate the tip at the required ultrasound frequency.

The intermediate portion 11 comprises a more rectilinear cylindrical portion 2 aligned with the proximal section 3 but having a smaller diameter. This cylindrical part 2 continues towards the distal part of the tip 1_{A} in a cylindrical part 4 of a smaller diameter forming a vertical bend 5 before being connected to the head 6.

The head 6 comprises a proximal section 9 having a cylindrical shape of the same diameter than the cylindrical part 4 which is progressively flattened and becomes thinner towards the distal part of the head 10.

This flat distal part 10 is as thin as possible with a thickness of at least 600 μm. It comprises a cutting head 8 having teeth in semi-circle which is intended to be brought into contact with the bone tissue to be cut or pierced and to penetrate the bones and a flattened part 7 which is not intended to penetrate inside the bones. The cutting head 8 will be visually distinct from the flattened part 7 to help the surgeon. The cutting head 8 will have the dimensions carefully chosen for making micro incision of between 3 and 15 mm depth and between 1 and 7 mm wide. The cutting head 8 is longitudinally oriented regarding the piezoelectric transducer axis of the ultrasonic surgical knife and vibrates in the sagittal plane regarding the piezoelectric transducer axis of the ultrasonic surgical knife. The form of the cutting head 8 (teeth in semi-circle) enable the adjustment of the instrument on the bones shapes (for examples of the jaw and of the mandible) in order to carried out the operation in a precise manner.

By offering a clear landmark on the head 8 to the practitioner, the tip 1_{A} will enable him to perform a less invasive act while stimulating the bones remodeling and therefore shortening the wound healing period. This tip will therefore enable the surgery act to be more precise, less invasive and more reproducible than the known tip currently available on the market even for an inexperienced practitioner.

Referring now to FIG. 12 there is illustrated a second type of piezo ultrasonic surgical knife tip (tip B) 1_{B}. This tip is identical to the tip 1_{A} except that the bend 5 is not vertical but horizontal and oriented to the right. Moreover the proximal section 9 of the head 6 is vertically bent with half torsion in order that on the right side view of the tip 1_{B}, it will be possible to see the front view of the head 6. This tip is used to do lateral maxillary or mandibular piezocision cuts. Here for the left side of the patient.

Referring now to FIG. 13 there is illustrated a third type of piezo ultrasonic surgical knife tip (tip C) 1_{c}. This tip is identical to the tip 1_{B} except that the bend 5 is oriented to the left in order that on the right side view of the tip 1_{C}, it will be possible to see the rear view of the head 6. This tip is used to do lateral maxillary or mandibular piezocision cuts. Here for the right side of the patient.

Referring now to FIG. 14 there is illustrated a fourth type of piezo ultrasonic surgical knife tip (tip D) 1_{D}. This tip is identical to the tip 1_{B} except that the bend 5 is longer. This tip is used for upper or lower molar distalization during piezocision as well as computer guided piezocision surgery

### EXAMPLES

The objective of the following example was to investigate

if using a piezo ultrasonic surgical knife with a ultrasonic frequency of between 28 000 and 36 000 Hz will have an impact on bone density and on bone volume when compared to bur surgery and
if so, the molecular mechanism of Piezocision and in particular if it is mediated or not by nitric oxide.
Therefore Wild-type (WT) and inducible nitric oxide (iNOS) knockout (KO) mice were utilized in this example.

Methods:

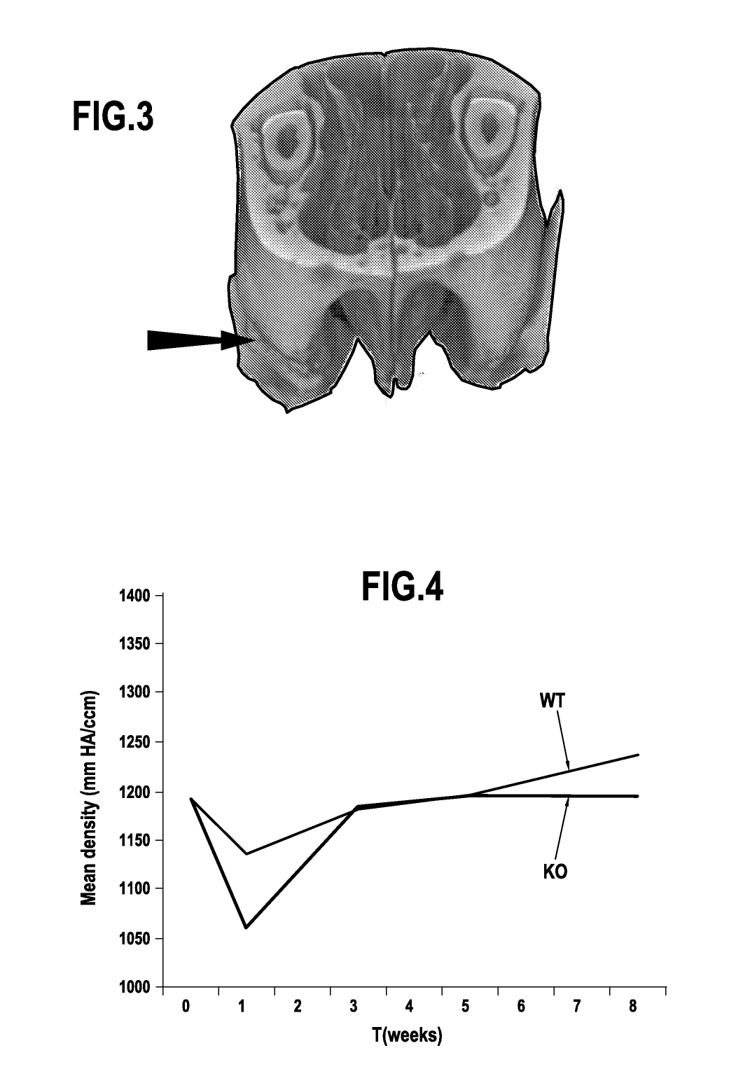

A total of eighty male mice (C57/BL6 wild type (WT) and iNOS KO (KO) mice) were purchased from Taconic (Germantown, N.Y.). Forty of these mice were WT and 40 were KO. Experimental procedures were approved by the Institutional Animal Care and Use Committee of Boston University.
Mice were anesthetized with ketamine/xylazine (100 mg/kg and 10 mg/kg, respectively). The mouse's jaw was held open with a custom-made retractor. Using a Piezotome®-with an tip BS1 (Satelec, France) a 1 mm deep and 0.5 mm wide lesion was created mesial to the 1st molar on the right side of the maxillary hard palate. The left side was left intact and served as control (FIG. 3). After 1, 3, 5 or 8 weeks WT and KO mice were sacrificed using carbon dioxide asphyxiation and the heads were collected for μCT analysis at 6 μm resolution (Scanco Medical μCT40).
In a separate set of animals (20 mice) bur osteotomy was performed in the same intraoral site using 1 mm diameter carbide round dental bur at 2000 rpm. Mice were sacrificed 8 weeks later.
Bone density (mg HA/ccm) and bone volume/total volume (BV/TV) data were collected.
Bone density was measured with micro CT and compared to hydroxyapatite (HA) standard, expressed as mg HA per cubic centimeter (mg HA/ccm). Bone volume/total volume (BV/TV) was measured using Micro CT Scan. Control values were measured on the contralateral untreated site of the maxilla at each time point and were pooled for WT and KO mice (0 time point). Mean and standard deviation is reported for each timepoint. P<0.05 was considered statistically significant.

### RESULTS

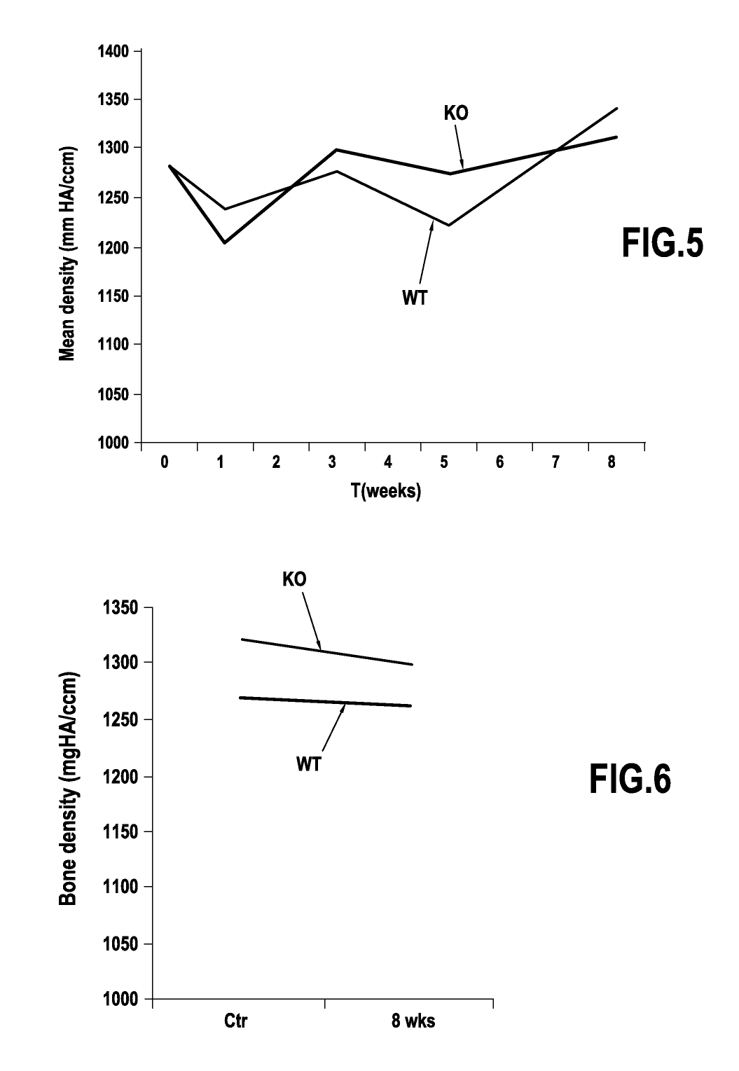

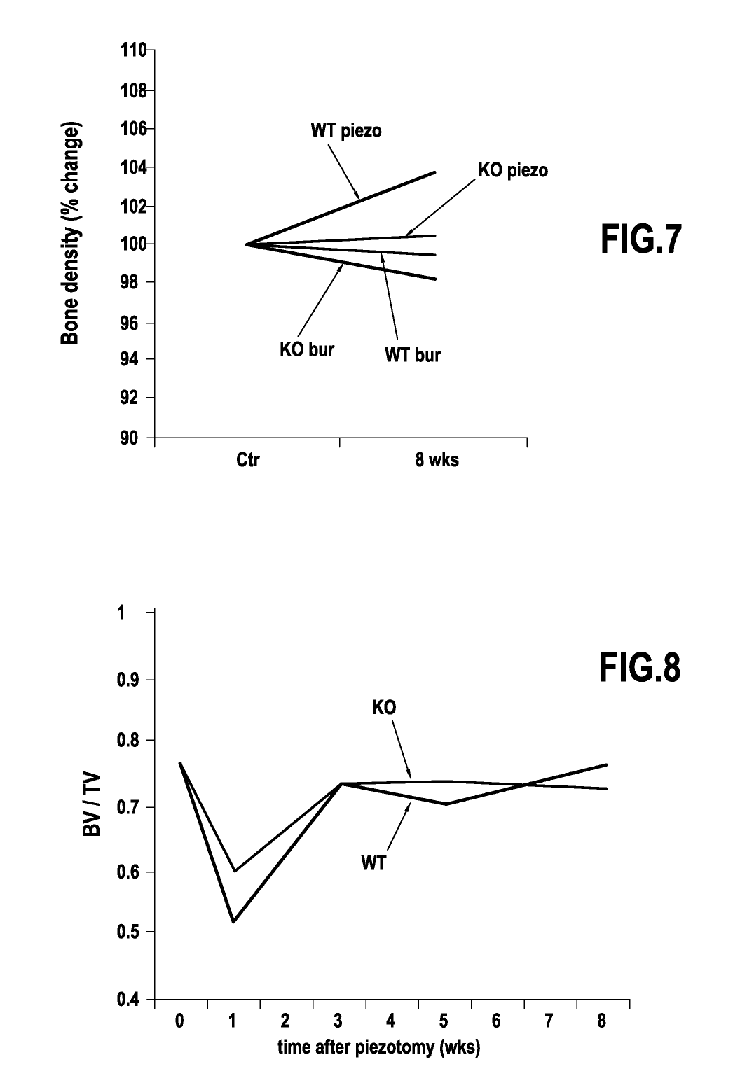

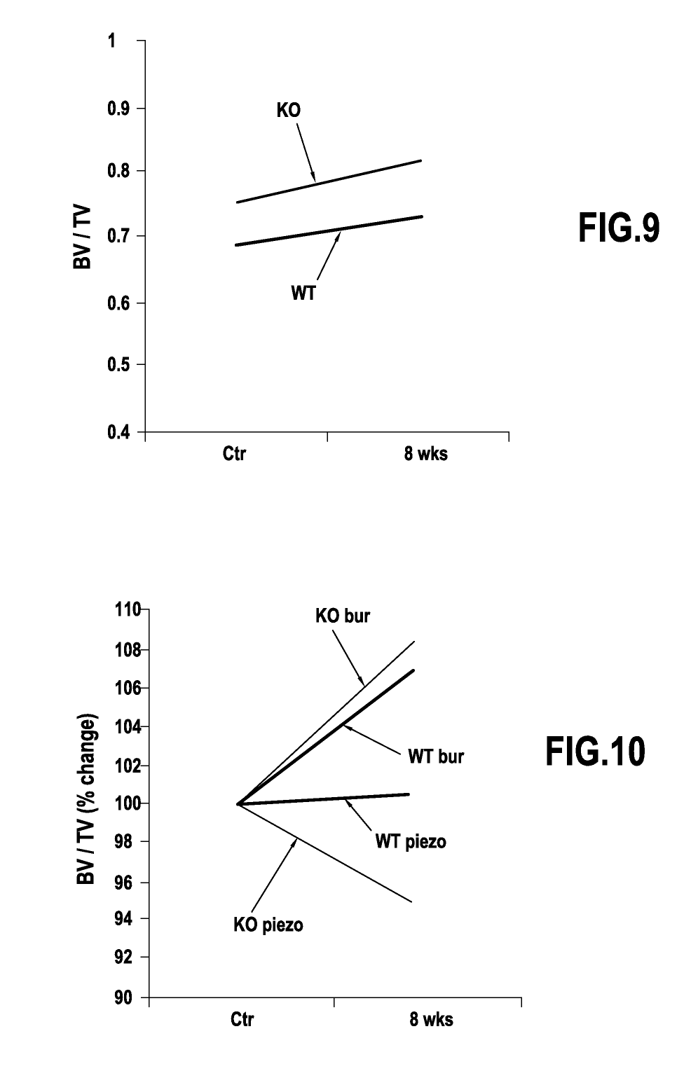

As can be seen in FIG. 4, one week after piezo-ultrasonic osteotomy there is a marked drop in bone density after piezo-ultrasonic osteotomy in both WT (5% decrease) and KO mice (11% decrease). By 3 weeks bone density returns to normal baseline in both mouse strains. Eight weeks after piezo cut there is an additional 4% increase in bone density in WT mice. This increase is not observed in KO mice. This increase is therefore significantly higher in WT mice (n=4-5 each time point, mean±SD: P<0.05 at 8 weeks). As can be seen in FIG. 5 this decrease and increase also occurred behind the cut site (at around 2-3 mm from the cut site). On the contrary, bur osteotomy did not induce significant change in bone density at 8 week timepoint in WT or KO mice (FIG. 6). Direct comparison of percent change in bone density (FIG. 7) shows that only piezo-ultrasonic osteotomy in WT mice induces an increase in bone density at 8 weeks.
Following piezo-ultrasonic osteotomy, BV/TV temporarily decreased at week 1 and recovered to control value at week 3 (FIG. 8). No significant changes were noted in BV/TV from week 3 to week 8 and no significant difference between WT and KO mice were found. Bur osteotomy on the other hand induced significant increase in BV/TV in KO mice after 8 weeks (FIG. 9). In WT mice, the increase in mean BV/TV in response to bur osteotomy did not reach statistical significance. Direct comparison of percent change in BV/TV (FIG. 10) shows that bur osteotomy induces increased BV/TV in KO mice at 8 weeks.

### Conclusions

Piezo-ultrasonic osteotomy significantly increases bone density while not affecting bone volume. Bur osteotomy on the contrary increases bone volume and does not affect bone density. Nitric oxide generated by iNOS mediates the piezo-ultrasonic osteotomy effect on bone density. Therefore, piezo-ultrasonic osteotomy increases bone density by a NO-dependent mechanism. In conclusion, in addition to its soft tissue-sparing properties, piezo-ultrasonic osteotomy may show advantages in surgical procedure where bone density is paramount.

1. Method for increasing bone density in a patient in need thereof which comprises the step of cutting the bones of said patient with a piezo ultrasonic surgical knife using a ultrasonic frequency of between 28 000 and 36 000 Hz.

2. The method of claim 1 wherein the patient in need thereof has bones of low quality.

3. The method according to claim 2 wherein the patient in need thereof is chosen between patients suffering from osteoporosis or diabetes, smokers, alcoholic, patients having type 3 or 4 bones, patients who are undergoing or have undergone long-term administration of corticosteroids, patients suffering from Cushing's syndrome, thyroidal or parathyroidal diseases or conditions and patients having gonadal dysgenesis.

4. The method according to claim 1 wherein the cutting consist in at least one micro incision of between 3 and 15 mm depth and between 1 and 7 mm wide.

5. The method according to claim 1 wherein the cutting of the bones occurred during surgery.

6. The method according to claim 5 wherein the surgery consists in surgery in which the bone density is paramount.

7. The method according to claim 6 wherein the surgery consists in oral surgery, maxillo facial surgery, implantology, veterinary application or orthopedic surgery.

8. The method of claim 6 wherein the surgery consists in implantology and the patient in need thereof has bones of low quality.

9. The method of claim 8 wherein the implantology consists in implant site preparation for dental implant with or without bone augmentation and the increase in bone density provides better implant primary stability and earlier prosthetic restoration.

10. The method of claim 7 wherein surgery consists in maxillo facial surgery.

11. The method of claim 10 wherein maxillo facial surgery consists in orthodontic tooth movement and the increase in bone density provides the avoidance of the use of any teeth retention means.
